(19) United States
(12) Patent Application Publication
(10) Pub. No.: US 2007/0010868 A1
(43) Pub. Date: Jan. 11, 2007

(54) Lumenally-active device

(75) Inventors: Bran Ferren, Beverly Hills, CA (US); W. Daniel Hillis, Encino, CA (US); Roderick A. Hyde, Livermore, CA (US); Muriel Y. Ishikawa, Livermore, CA (US); Edward K.Y. Jung, Bellevue, WA (US); Nathan P. Myhrvold, Medina, WA (US); Elizabeth A. Sweeney, Seattle, WA (US); Clarence T. Tegreene, Bellevue, WA (US); Richa Wilson, San Francisco, CA (US); Lowell L. Wood JR., Livermore, CA (US); Victoria Y.H. Wood, Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 11/478,368

(22) Filed: Jun. 28, 2006

Related U.S. Application Data
(63) Continuation-in-part of application No. 10/949,186, filed on Sep. 24, 2004.
(63) Continuation-in-part of application No. 10/827,576, filed on Apr. 19, 2004.
(63) Continuation-in-part of application No. 10/827,578, filed on Apr. 19, 2004.
(63) Continuation-in-part of application No. 10/827,572, filed on Apr. 19, 2004.
(63) Continuation-in-part of application No. 10/827,390, filed on Apr. 19, 2004.
(63) Continuation-in-part of application No. 11/403,230, filed on Apr. 12, 2006.

(51) Int. Cl. A61N 1/00; A61F 2/06

(52) U.S. Cl. 623/1.15; 607/2; 623/1.1

(57) ABSTRACT

Embodiments of a lumenally-active system and method of use and control thereof are disclosed. According to various embodiments, a lumenally-active device is positioned in a body lumen of an organism, where the device may sense a parameter of a fluid in the body lumen and perform an action on the fluid. Control logic and/or circuitry may be located on the device, or the system may include a separate control module. Liquid or gaseous fluids may be treated by embodiments of the device. Actions may include, for example, modification of a body fluid by addition or removal of a material, or by modification of a property of a body fluid or a component thereof.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation-in-part of U.S. patent application Ser. No. 10/949,186, entitled A CILIATED STENT-LIKE SYSTEM, naming Richa Wilson, Victoria Y. H. Wood, W. Daniel Hillis, Clarence T. Tegreene, Muriel Y. Ishikawa, and Lowell L. Wood, Jr. as inventors, filed 24 Sep. 2004, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 10/827,576, entitled A SYSTEM FOR PERFUSION MANAGEMENT, naming Lowell L. Wood, Jr. as inventor, filed 19 Apr. 2004, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 10/827,578, entitled A SYSTEM WITH A SENSOR FOR PERFUSION MANAGEMENT, naming Lowell L. Wood, Jr. as inventor, filed 19 Apr. 2004, which is currently co-pending, or is an application of which a currently co-pending application is entitled to the benefit of the filing date. For purposes of the USPTO extra-statutory requirements, the present application constitutes a continuation-in-part of U.S. patent application Ser. No. 10/827,572, entitled A SYSTEM WITH A RESERVOIR FOR PERFUSION MANAGEMENT, naming Lowell L. Wood, Jr. as inventor, filed 19 Apr. 2004, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 10/827,390, entitled A TELESCOPING PERFUSION MANAGEMENT SYSTEM, naming Lowell L. Wood, Jr. as inventor, filed 19 Apr. 2004, which is currently co-pending, or is an application of which a currently co-pending application is entitled to the benefit of the filing date. The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present applicant entity has provided above a specific reference to the application(s) from which priority is being claimed as recited by statute. Applicant entity understands that the statute is unambiguous in its specific reference language and does not require either a serial number or any characterization, such as “continuation” or “continuation-in-part,” for claiming priority to U.S. patent applications. Notwithstanding the foregoing, applicant entity understands that the USPTO's computer programs have certain data entry requirements, and hence applicant entity is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### BACKGROUND

Devices and systems have been developed for use in various body lumens, particularly in the cardiovascular system, digestive, and urogenital tract. Catheters are used for performing a variety of sensing and material delivery tasks. Stents are implanted in blood vessels for the purpose of preventing stenosis or restenosis of blood vessels. Capsules containing sensing and imaging instrumentation that may be swallowed by a subject and which travel passively through the digestive tract have also been developed. Robotic devices intended to move through the lower portion of the digestive tract under their own power are also under development.

### SUMMARY

The present application describes devices, systems, and related methods for treatment of fluid in a body lumen. Embodiments of lumenally-active devices for placement in body lumens are disclosed. In one aspect, a system includes but is not limited to a sensor, response initiation circuitry, and an active portion capable of performing an action. In addition to the foregoing, other system aspects are described in the claims, drawings, and text forming a part of the present disclosure.

In one aspect, a method includes but is not limited to detecting a condition of interest in a fluid within a body lumen, generating a response initiation signal with response initiation circuitry, and performing an action within the body lumen with the active portion of the lumenally-active device. In addition to the foregoing, other method aspects are described in the claims, drawings, and text forming a part of the present disclosure.

Various aspects of the operation of such lumenally-active devices may be performed under the control of hardware, software, firmware, or a combination thereof. In one or more various aspects, related systems include but are not limited to circuitry and/or programming for effecting the herein-referenced method aspects; the circuitry and/or programming can be virtually any combination of hardware, software, and/or firmware configured to effect the herein referenced method aspects depending upon the design choices of the system designer. Software for operating a lumenally-active device according to various embodiments is also described.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is an illustration of an embodiment of a lumenally-active device;

FIGS. 2A-2D are illustrations of several embodiments of lumenally-active device structures;

FIGS. 3A-3C are illustrations of several embodiments of materials for lumenally-active device structures;

FIGS. 4A and 4B are illustrations of a device structure having a variable length and diameter;

FIGS. 5A-5F are cross-sectional views of a number of embodiments of lumenally-active device structures;

FIG. 6 is a schematic diagram of a lumenally-active device;

FIGS. 7A and 7B are longitudinal cross-sectional views of the treatment of a fluid flowing through a lumenally-active device;

FIGS. 8A-8D are illustrations of several embodiments of lumenally-active device active portions;

FIGS. 9A and 9B are illustrations of several further embodiments of lumenally-active device active portions;

FIG. 10 is a depiction of a lumenally-active device including a fluid structure;

FIG. 11 is a depiction of a lumenally-active device including a material collection structure;

FIG. 12 is an illustration of a device including stored deliverable material;

FIG. 13 is a cross-sectional view of an embodiment of a device including a stored deliverable material and a barrier release mechanism;

FIG. 14 is a cross-sectional view of another embodiment of a device including a stored deliverable material and a barrier release mechanism;

FIGS. 15A and 15B are depictions of the release of a stored deliverable material from a reservoir via a rupturable barrier;

FIGS. 16A and 16B are depictions of the release of a stored deliverable material from a reservoir via a degradable barrier;

FIGS. 17A and 17B are depictions of the release of a stored deliverable material from a reservoir via a barrier having controllable permeability;

FIG. 18 is a cross-sectional view of another embodiment of a device including a stored deliverable material;

FIGS. 19A and 19B are depictions of the release of a stored deliverable material from a carrier material;

FIG. 20 is an illustration of a lumenally-active system that includes an external control portion;

FIG. 21 is a block diagram of a device depicting various alternative and/or optional components;

FIG. 22 is a flow diagram of a method of treating a body fluid; and

FIG. 23 is schematic diagram of a system including software modules.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

According to various embodiments described herein, a lumenally-active system may include a structural element configured to fit within at least a portion of a body lumen, the structural element including a lumen-wall-engaging portion and a fluid-contacting portion configured to contact fluid within the body lumen; a sensor capable of detecting a condition of interest in the fluid; response initiation circuitry operatively connected to the sensor and configured to generate a response initiation signal upon detection of the condition of interest in the fluid by the sensor; and an active portion operatively connected to the response initiation circuitry and capable of producing a response upon receipt of the response initiation signal.

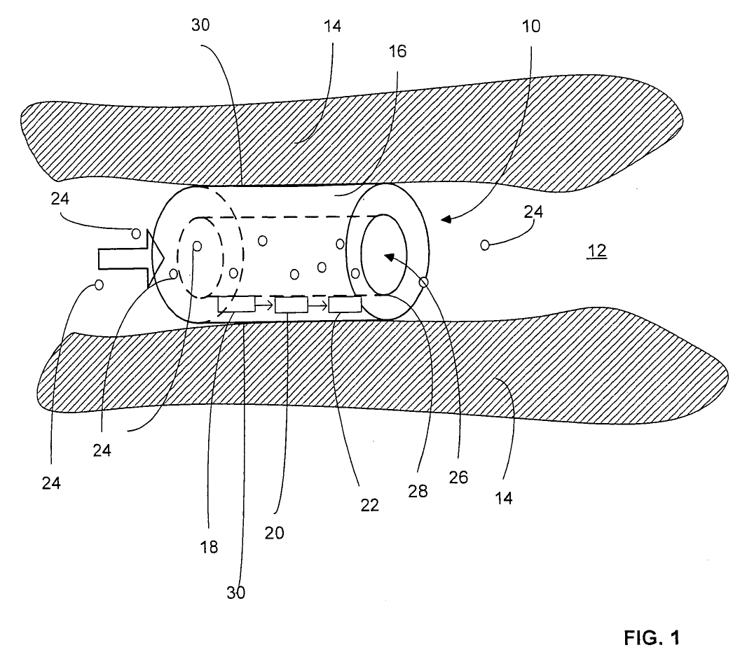

FIG. 1 depicts a first embodiment of a lumenally-active device 10 positioned in a body lumen 12. Body lumen 12 is defined by wall portions 14, which may be the walls of a blood vessel or other lumen-containing structure within the body of an organism. Lumenally-active device 10 includes structural element 16, sensor 18, response initiation circuitry 20, and active portion 22. In this example, a body fluid flows through lumen 12 in the direction indicated by the arrow. Body fluid components 24, which may be, for example, cells, cellular fractions or components, collections or aggregations of cells, bacterial, viral or fungal species, ions, molecules, gas bubbles, dissolved gas, suspended particles, or a variety of other materials that may be present in the body fluid, are also indicated. Body fluid components may be materials that are normally present in the body fluid, materials that are naturally derived but not normally present in the body fluid, or foreign materials that have entered or been introduced to the body fluid (including but not limited to pathogens, toxins, pollutants, or medications, for example). Fluid flows through the central opening 26 of structural element 16, with the interior surface of structural element 16 forming fluid-contacting surface 28. In the embodiment of FIG. 1, sensor 18 and active portion 22 may be located at a fluid-contacting surface 28. In the embodiment of FIG. 1, outer surface 30 of structural element 16 functions as a lumen-wall engaging portion, providing a frictional fit with wall portions 14. In other embodiments of lumenally-active devices, other structures and methods for engaging the lumen wall may be employed.

Embodiments of the lumenally-active system may be configured for use in various different body lumens of an organism including, for example, a nostril or nasal cavity, the respiratory tract, the cardiovascular system (e.g., a blood vessel), the lymphatic system, the biliary tract, the urogenital tract, the oral cavity, the digestive tract, the tear ducts, a glandular system, a reproductive tract, the cerebral ventricles, spinal canal, and other fluid-containing structure of the nervous system of an organism. Other fluid-containing lumens within the body may be found in the auditory or visual system, or in interconnections thereof, e.g., the Eustachian tubes. Although many of the devices and systems described herein may be used in body lumens through which fluid flows, it is not intended that such devices or systems are limited to use in tubular lumen-containing structures containing moving fluid; in some applications a lumenally-active device may be placed in a body lumen containing relatively unmoving, or intermittently moving fluid. Wherever a lumenally-active device or system is to be used, the dimensions and mechanical properties (e.g., rigidity) of the lumenally-active system, and particularly of the structural element of the lumenally-active system, may be selected for compatibility with the location of use, in order to provide for reliable positioning of the device and to prevent damage to the lumen-containing structure including the body lumen.

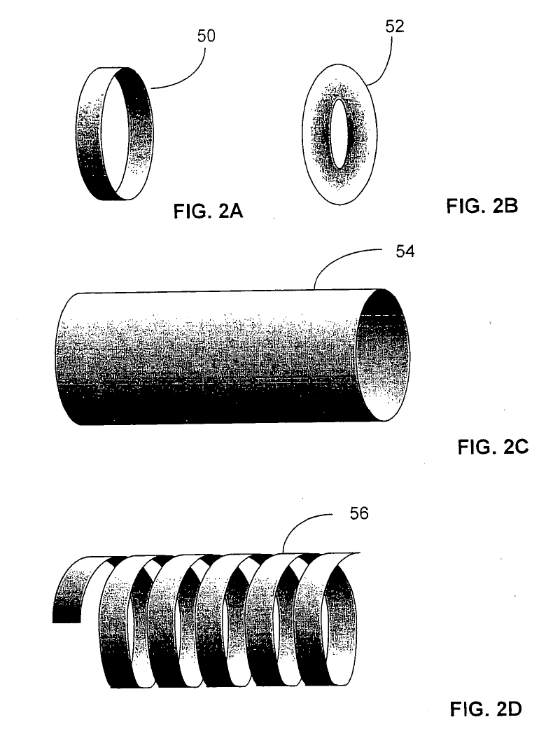

FIGS. 2A-2D depict a number of possible configurations for structural elements of lumenally-active devices for use in body lumens. Structural elements may have the form of a short cylinder 50, as shown in FIG. 2A; an annulus 52, as shown in FIG. 2B; a cylinder 54, as shown in FIG. 2C; or a spiral 56, as shown in FIG. 2D. Elongated forms such as cylinder 54 or spiral 56 may be suitable for use in tubular lumen-containing structures such as, for example, blood vessels. Structural elements may be formed from various materials, including metals, polymers, fabrics, and various composite materials, including ones of either inorganic or organic character, the latter including materials of both biologic and abiologic origin, selected to provide suitable biocompatibility and mechanical properties.

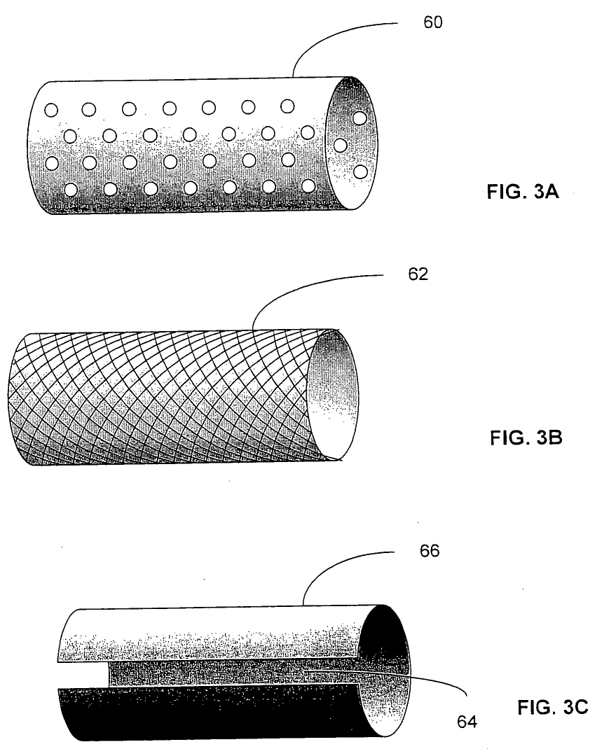

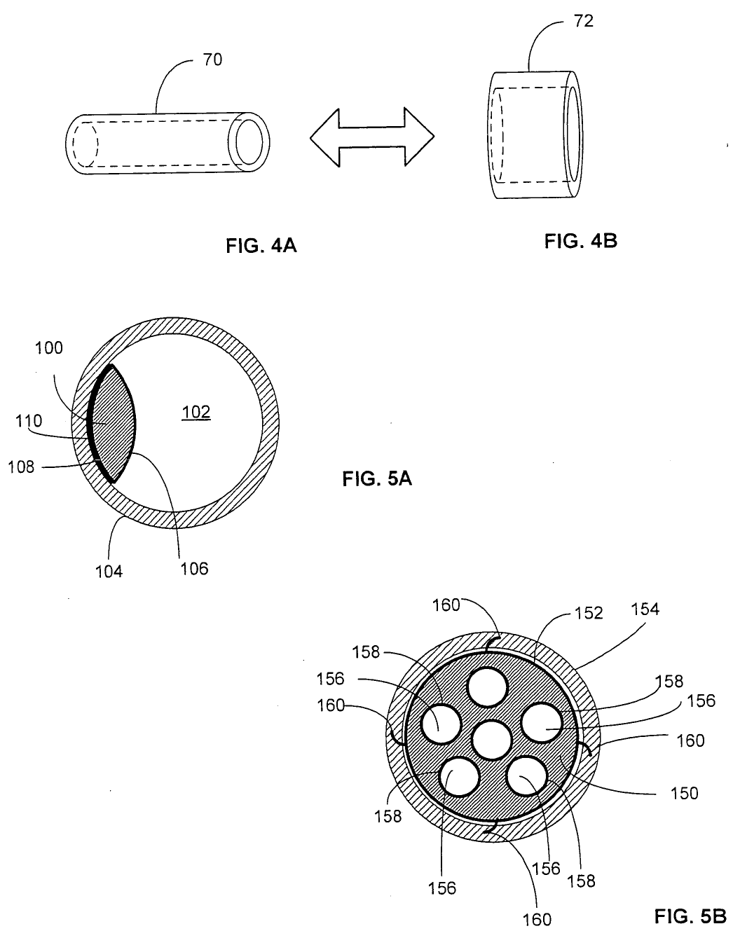

As shown in FIGS. 3A-3C, the basic form of a structural element may be subject to different variations, e.g., by perforations, as shown in structural element 60 in FIG. 3A; a mesh structure, as shown in structural element 62 in FIG. 3B; or the inclusion of one or more slots 64 in structural element 66 in FIG. 3C. Slot 64 runs along the entire length of structural element 66; in other embodiments, one or more slots (or mesh or perforations) may be present in only a portion of the structural element. By using spiral, mesh, or slotted structural elements (as in FIGS. 2D, 3B, and 3C) formed from resilient, elastic, springy or self-expanding/self-contracting structural elements may be formed. A self expanding or contracting structural element may facilitate positioning of the structural element within a body lumen of an organism. In some embodiments, flexible material having adjustable diameter, taper, and length properties may be used. For example, some materials may change from a longer, narrower configuration 70 as shown in FIG. 4A, to a shorter, wider configuration 72 as shown in FIG. 4B, or may taper over their length. Structural elements that may exhibit this type of expansion/contraction property may include mesh structures formed of various metals or plastics, and some polymeric materials, for example.

The exemplary embodiments depicted in FIGS. 2A-2C, 3A-3C, and 4A and 4B are substantially cylindrical, and hollow and tubular in configuration, with a single central opening. Thus, the exterior of the cylindrical structural element may contact and engage the wall of the body lumen, and the interior of the structural element (within the single central opening) may form the fluid-contacting portion of the structural element. Lumenally-active devices according to various embodiments are not limited to cylindrical structural elements having a single central opening, however.

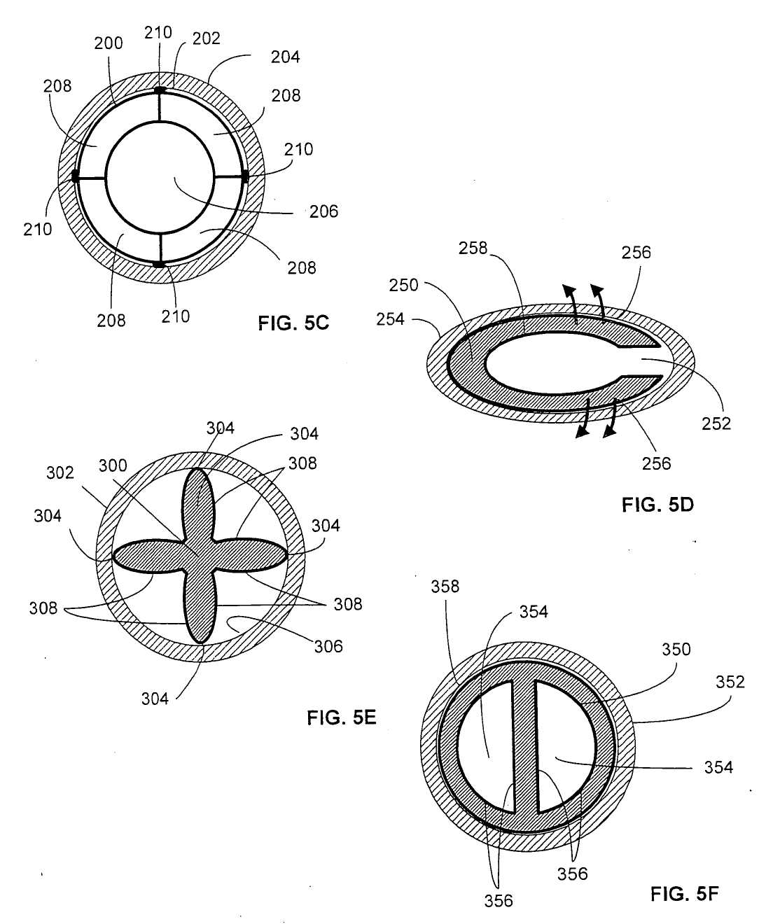

FIGS. 5A through 5F depict a variety of cross-sectional configurations for structural elements of lumenally-active devices. In FIG. 5A, a lumenally-active device 100 is positioned in lumen 102 of lumen-containing structure 104. In this embodiment, fluid-contacting portion 106 may be the surface of structural element 100 that faces lumen 102, while the lumen-wall engaging portion 108 may be a layer of tissue adhesive on surface 110 of structural element 100.

FIG. 5B depicts in cross-section a further embodiment of a structural element 150 in lumen 152 of lumen-containing structure 154. Structural element 150 includes multiple openings 156, each of which includes an interior surface 158 that forms a fluid-contacting portion. Structural element 150 may include one or more barb-like structures 160 that serve as lumen-wall engaging portions that maintain structural element 150 in position with respect to lumen-containing structure 154.

FIG. 5C depicts in cross-section an embodiment of a structural element 200 in lumen 202 of lumen-containing structure 204. Structural element 200 includes a large central opening 206 and multiple surrounding openings 208. The interior surface of each opening 206 or 208 serves as a fluid-contacting portion, while projections 210 function as lumen-wall engaging portions, which may engage frictionally or may project slightly into the interior of the wall of lumen-containing structure 204.

FIG. 5D depicts a further embodiment in which structural element 250 has a substantially oval cross-section and includes a slot 252. Lumen-containing structure 254 may be generally oval in cross section, or may be flexible enough to be deformed to the shape of structural element 250. Structural element 250 may be a compressed spring-like structure that produces outward forces as indicated by the black arrows, so that end portions 256 of structural element 250 thus press against and engage the lumen wall. Interior surface 258 of structural element 250 serves as the fluid-contacting portion of structural element 250.

FIG. 5E is a cross-sectional view of a structural element 300 in a lumen-containing structure 302. Structural element 300 includes multiple projecting arms 304 which contact lumen wall 306 of lumen-containing structure 302, and function as lumen-wall engaging portions. Inner surfaces 308 of arms 304 function as fluid-contacting portions of structural element 300.

FIG. 5F depicts (in cross-section) another example of a structural element 350 positioned within a lumen-containing structure 352. Structural element 350 includes two openings 354. The interior surfaces 356 of openings 354 function as fluid-contacting portions, while the outer surface 358 of structural element 350 serves as a lumen-wall engaging portion.

The structural elements depicted in FIGS. 2-5 are intended to serve as examples, and are in no way limiting. The choice of structural element size and configuration appropriate for a particular body lumen may be selected by a person of skill in the art. Structural elements may be constructed by a variety of manufacturing methods, from a variety of materials. Appropriate materials may include metals, ceramics, polymers, and composite materials having suitable biocompatibility, sterilizability, mechanical, and physical properties, as will be known to those of skill in the art. Examples of materials and selection criteria are described, for example, in The Biomedical Engineering Handbook, Second Edition, Volume I, J. D. Bronzino, Ed., Copyright 2000, CRC Press LLC, pp. IV-1-43-31. Manufacturing techniques may include injection molding, extrusion, die-cutting, rapid-prototyping, etc., and will depend on the choice of material and device size and configuration. Sensing and active portions of the lumenally-active device as well as associated electrical circuitry (not depicted in FIGS. 2-5) may be fabricated on the structural element using various microfabrication and/or MEMS techniques, or may be constructed separately and subsequently assembled to the structural element, as one or more distinct components.

The term fluid, as used herein, may refer to liquids, gases, and other compositions, mixtures, or materials exhibiting fluid behavior. The fluid within the body lumen may include a liquid, or a gas or gaseous mixtures. As used herein, the term fluid may encompass liquids, gases, or mixtures thereof that also include solid particles in a fluid carrier. Liquids may include mixtures of two or more different liquids, solutions, slurries, or suspensions. Examples of liquids present within body lumens include blood, lymph, serum, urine, semen, digestive fluids, tears, saliva, mucous, cerebro-spinal fluid, intestinal contents, bile, epithelial exudate, or esophageal contents. Liquids present within body lumens may include synthetic or introduced liquids, such as blood substitutes or drug, nutrient, or buffered saline solutions. Fluids may include liquids containing dissolved gases or gas bubbles, or gases containing fine liquid droplets or solid particles. Gases or gaseous mixtures found within body lumens may include inhaled and exhaled air, e.g. in the nasal or respiratory tract, or intestinal gases.

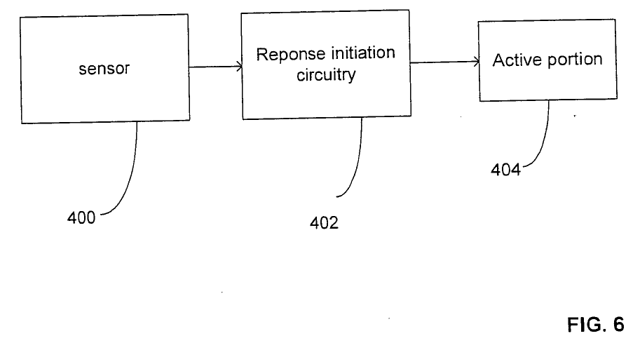

FIG. 6 is a schematic diagram of a lumenally-active device, including sensor 400, response initiation circuitry 402, and active portion 404. Sensor 400 may be used to detect a condition of interest in the fluid, which may include, for example, detecting pressure, temperature, fluid flow, presence of a cell of interest, or concentration of a chemical or chemical species (including ionic species) of interest. Sensor 400 may sense a wide variety of physical or chemical properties. In some embodiments, detecting a condition of interest may include detecting the presence (or absence) of a material or structure of interest in the fluid. Sensor 400 may include one or more of an optical sensor, an imaging device, an acoustic sensor, a pressure sensor, a temperature sensor, a flow sensor, a viscosity sensor, or a shear sensor for measuring the effective shear modulus of the fluid at a frequency or strain-rate, a chemical sensor for determining the concentration of a chemical compound or species, a biosensor, or an electrical sensor, for example. An optical sensor may be configured to measure the optical absorption, optical emission, fluorescence, or phosphorescence of at least a portion of the fluid of the fluid, for example. Such optical properties may be inherent optical properties of all or a portion of the fluid, or may be optical properties of materials added or introduced to the fluid, such as tags or markers for materials of interest within the fluid. A biosensor may detect materials including, but not limited to, a biological marker, an antibody, an antigen, a peptide, a polypeptide, a protein, a complex, a nucleic acid, a cell (and, in some cases, a cell of a particular type, e.g. by methods used in flow cytometry), a cellular component, an organelle, a gamete, a pathogen, a lipid, a lipoprotein, an alcohol, an acid, an ion, an immunomodulator, a sterol, a carbohydrate, a polysaccharide, a glycoprotein, a metal, an electrolyte, a metabolite, an organic compound, an organophosphate, a drug, a therapeutic, a gas, a pollutant, or a tag. A biosensor may include an antibody or other binding molecule such as a receptor or ligand. Sensor 400 may include a single sensor or an array of sensors, and is not limited to a particular number or type of sensors. Sensor 400 might comprise in part or whole, a gas sensor such as an acoustic wave, chemiresistant, or piezoelectric sensor, or perhaps an electronic nose. Sensor 400 may be very small, comprising a sensor or array that is a chemical sensor (Chemical Detection with a Single-Walled Carbon Nanotube Capacitor E. S. Snow, 2005 Science Vol. 307; 1942-1945), a gas sensor (Smart single-chip gas sensor microsystem Hagleitner, C. et al. 2001 NATURE VOL 414 p. 293-296.), an electronic nose, a nuclear magnetic resonance imager (“Controlled multiple quantum coherences of nuclear spins in a nanometre-scale device”, Go Yusa, 2005, Nature 343: 1001-1005). Further examples of sensors are provided in The Biomedical Engineering Handbook, Second Edition, Volume 1, J. D. Bronzino, Ed., Copyright 2000, CRC Press LLC, pp. V-1-51-9, and U.S. Pat. No. 6,802,811, both of which are incorporated herein by reference. A sensor may be configured to measure various parameters, including, but not limited to, the electrical resistivity of the fluid, the density or sound speed of the fluid, the pH, the osmolality, or the index of refraction of the fluid at at least one wavelength. The selection of a suitable sensor for a particular application or use site is considered to be within the capability of a person having skill in the art.

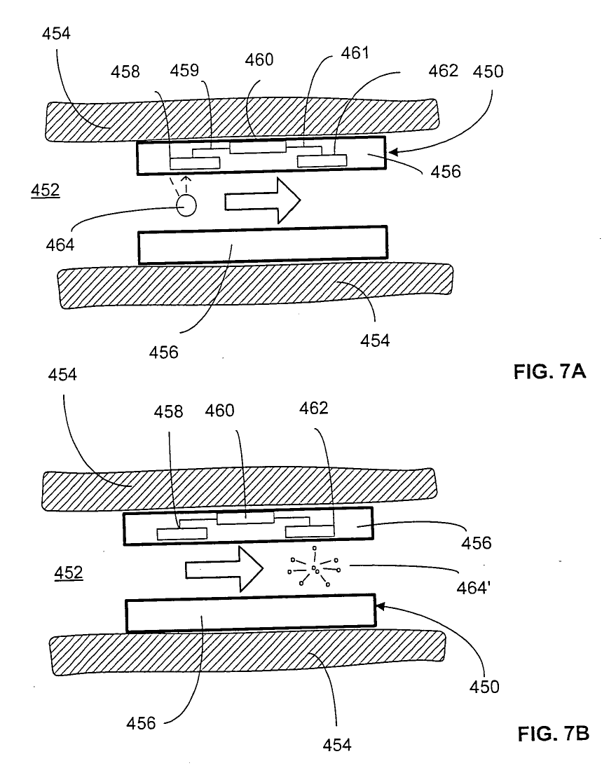

FIGS. 7A and 7B illustrate the treatment of a fluid flowing through a lumenally-active device similar to that shown in FIG. 1. Body lumen 452 is defined by wall portions 454. In FIG. 7A, component 464 of fluid flowing through body lumen 452 is detected by sensor 458 in structural element 456 of lumenally-active device 450. Upon detection of component 464 by sensor 458, a sense signal 459 is sent to response initiation circuitry 460, which generates a response initiation signal 461. Response initiation signal 461 is sent to active portion 462. As shown in FIG. 6B, upon receipt of response initiation signal 461, active portion 462 produces a response or action, which in this example is a pulse of energy (e.g. acoustic energy) to destroy or component 464 (indicated following destruction by reference number 464′). For example, a pulse of acoustic energy may be used to modify a kidney stone in the urinary tract, or to modify another object in another body fluid.

In some applications, detecting a condition of interest in the fluid within the body lumen may include detecting the presence of a material of interest in the fluid within the body lumen. A material of interest in a fluid may include, for example, an object such as a blood clot, a thrombus, an embolus, a plaque, a lipid, a kidney stone, a dust particle, a pollen particle, an aggregate, a cell, a specific type of cell, a cellular component, an organelle, a collection or aggregation of cells or components thereof, a gamete, a pathogen, or a parasite.

In connection with detection of the presence of a material of interest in the fluid within the body lumen, the active portion of the lumenally-active system may be capable of removing, modifying, or destroying the material of interest. Modification or destruction of the material of interest may be accomplished by the release of a suitable material (e.g. an anti-coagulant for destroying a blood clot, complement to coat a parasite for recognition by the immune system, or by the release of an anti-inflammatory, biomimetic or biologic to bind to and inactivate an inflammatory mediator such as fNFa, by the delivery of suitable energy (e.g., acoustic energy for modifying a kidney stone, electromagnetic energy such as light to cause a photoreaction, break bonds in a molecule, produce heating, etc., or by delivery of heat or cold or other chemo-physical change (e.g. ambient pressure, pH, osmolality, toxic material introduction/generation) for tissue modification, as in ablation of circulating tumor cells or plaque or temperature-induced modification of sperm as it passes through the vas deferens.

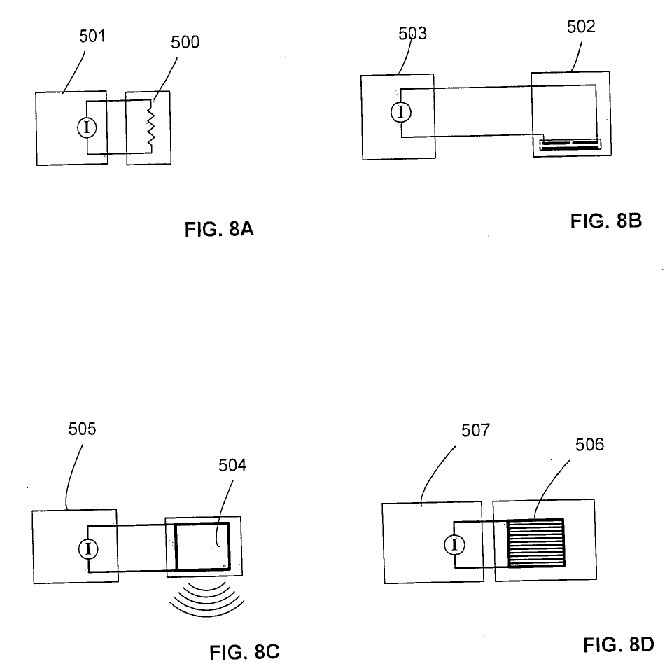

The lumenally-active device may include an active portion capable of producing a response upon receipt of the response initiation signal. FIGS. 8A-8D, 9A and 9B, and 10 and 11 provide examples of different active portions which may be included in a lumenally-active device. The active portion may include a heating element 500 as depicted in FIG. 8A, operatively coupled to the response initiation circuitry 501 and configured to produce heating in response to detection of the condition of interest. The heating element may be a resistive element that produces heat when current is passed through it, or it may be a magnetically active material that produces heat upon exposure to an electromagnetic field. Examples of magnetically active materials include permanently magnetizable materials, ferromagnetic materials such as iron, nickel, cobalt, and alloys thereof, ferrimagnetic materials such as magnetite, ferrous materials, ferric materials, diamagnetic materials such as quartz, paramagnetic materials such as silicate or sulfide, and antiferromagnetic materials such as canted antiferromagnetic materials which behave similarly to ferromagnetic materials; examples of electrically active materials include ferroelectrics, piezoelectrics and dielectrics. Alternatively, the active portion may include a cooling element 502 as depicted in FIG. 8B, operatively coupled to the response initiation circuitry 503 and configured to produce cooling in response to detection of the condition of interest. Cooling may be produced by a number of mechanisms and/or structures. For example, cooling may be produced by an endothermic reaction (such as the mixing of ammonium nitrate and water) initiated by opening of a valve or actuation of a container in response to a control signal. Other methods and/or mechanisms of producing cooling may include, but are not limited to, thermoelectric (Peltier Effect) and liquid-gas-vaporization (Joule-Thomson) devices.

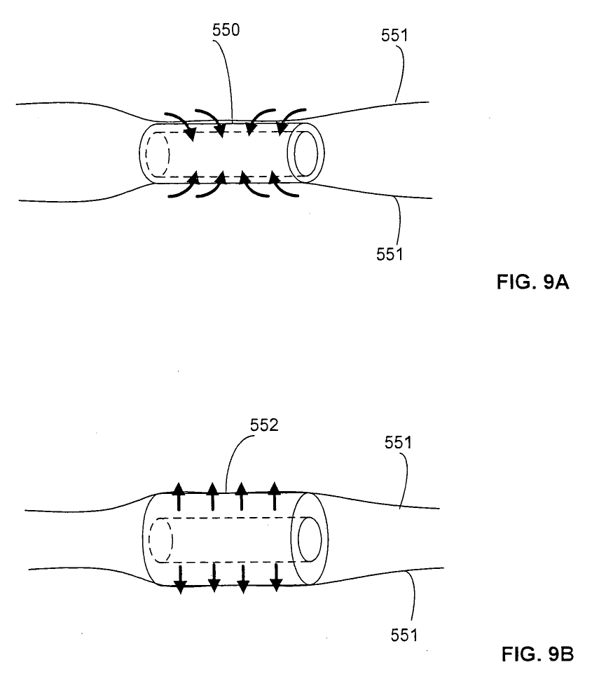

In some embodiments, the active portion may include an electromagnetic radiation source 504 as depicted in FIG. 8C, operatively coupled to the response initiation circuitry 505 and configured to emit electromagnetic radiation in response to detection of the condition of interest. Electromagnetic radiation sources may include light sources, for example, such as light emitting diodes and laser diodes, or sources of other frequencies of electromagnetic energy or radiation, radio waves, microwaves, ultraviolet rays, infra-red rays, optical rays, terahertz beams, and the like. In some embodiments, the active portion may include an electric field source or a magnetic field source. As another alternative, the active portion may include an acoustic energy source 506 (e.g., a piezoelectric crystal) as depicted in FIG. 8D, operatively coupled to the response initiation circuitry 507 and configured to emit acoustic energy in response to detection of the condition of interest. The active portion may include a pressure source operatively coupled to the response initiation circuitry and configured to apply pressure to a portion of the body lumen in response to detection of the condition of interest. Pressure source may include materials that expand through absorption of water, or expand or contract due to generation or consumption of gas or conformation changed produced by chemical reactions or temperature changes, electrically-engendered Maxwell stresses, osmotic stress-generators, etc. FIG. 9A depicts a negative pressure source 550 capable of applying negative pressure (in this example, substantially radially-inward force) to lumen walls 551, while FIG. 9B depicts a positive pressure (expanding or expansion) source 552, capable of applying positive pressure (in this example, a substantially radially-outward force) to lumen walls 551.

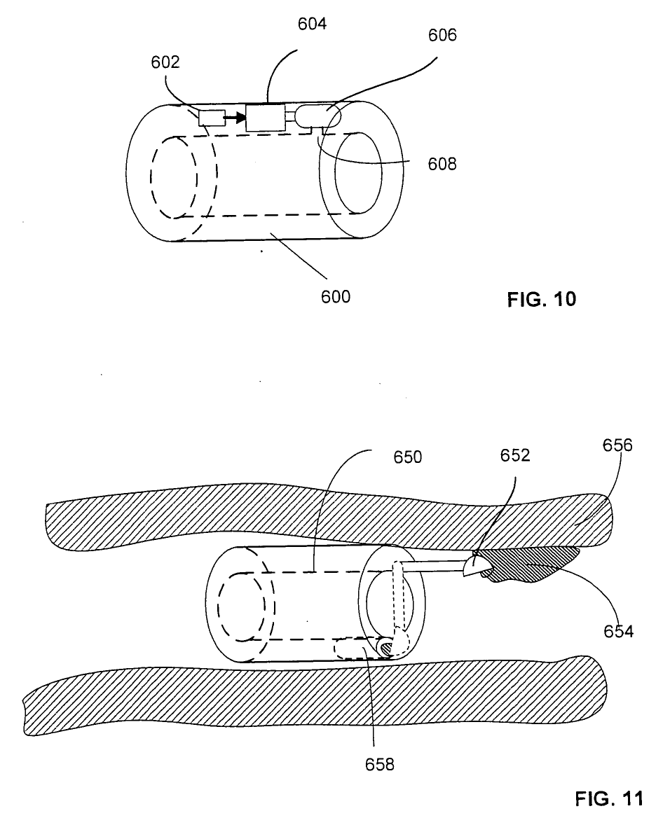

Alternatively, or in addition, in some embodiments the active portion may include a capture portion operatively coupled to the response initiation circuitry and configured to capture the detected material of interest. FIG. 10 depicts a device 600 including a fluid capture portion 606. Lumenally-active device 600 includes sensor 602, response initiation circuitry 604, and fluid capture portion 606. Fluid enters fluid capture portion 606 via inlet 608. Fluid capture portion 606 may be a reservoir, for example, into which fluid is drawn by capillary action. Alternatively, fluid may be pumped into capture portion 606. Captured fluid may be treated and released, or simply stored. In some applications, stored fluid may be subjected to analysis.

FIG. 11 depicts lumenally-active device 650 including a sample collection structure 652 capable of collecting a solid sample 654. In the example depicted in FIG. 11, solid sample 654 is a solid material found upon or immediately under the surface of the lumen-defining wall 656 (an arterial plaque, for example). Solid sample 654 placed in storage reservoir 658 by sample collection structure 652. In a related alternative embodiment, a lumenally-active device may include a filter or selective binding region to remove materials from fluid moving past or through the lumenally-active device.

In other embodiments, the active portion of a lumenally-active device may include a material release structure operatively coupled to the response initiation circuitry and configured to release a material in response to detection of a condition of interest.

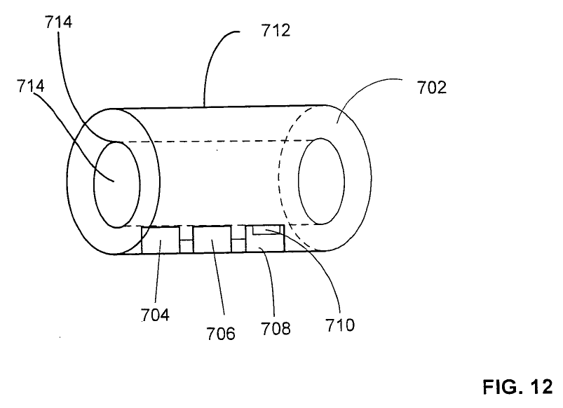

FIG. 12 depicts a lumenally-active device 700 including a structural element 702, sensor 704, response initiation circuitry 706, and material release structure 708 including release mechanism 710. Structural element 702 includes external surface 712, configured to fit within a body lumen, and internal surface 714 defining central opening 716, through which a fluid may flow. Upon sensing of a condition of interest in the fluid by sensor 704, response initiation circuitry 706 may cause release of material from material release structure 708 by activating release mechanism 710. Release mechanism 710 may include a variety of different types of release mechanisms.

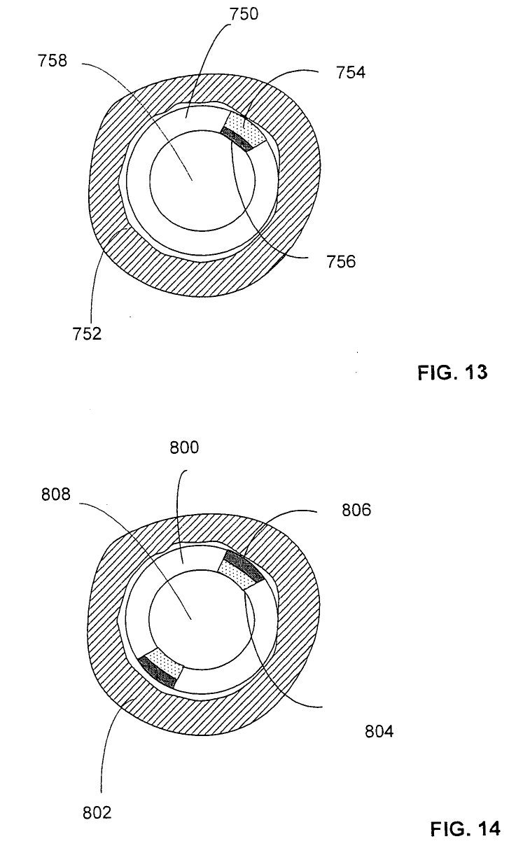

FIG. 13 illustrates, in cross sectional view, a structural element 750 of a lumenally-active device positioned in a lumen-containing structure 752. A reservoir 754 contains stored deliverable material. Barrier 756 is a controllable barrier that control the release of the stored deliverable material into central opening 758, and thus into a fluid that fills and/or flows through lumen-containing structure 752.

FIG. 14 illustrates an embodiment similar to that depicted in FIG. 13, including a structural element 800 of a lumenally-active device positioned in a lumen-containing structure 802. A reservoir 804 contains stored deliverable material. Barrier 806 is a controllable barrier that controls the release of the stored deliverable material. In the embodiment of FIG. 14, activation of barrier 806 causes release of the stored deliverable material toward the lumen wall of lumen-containing structure 802, rather than into central opening 808.

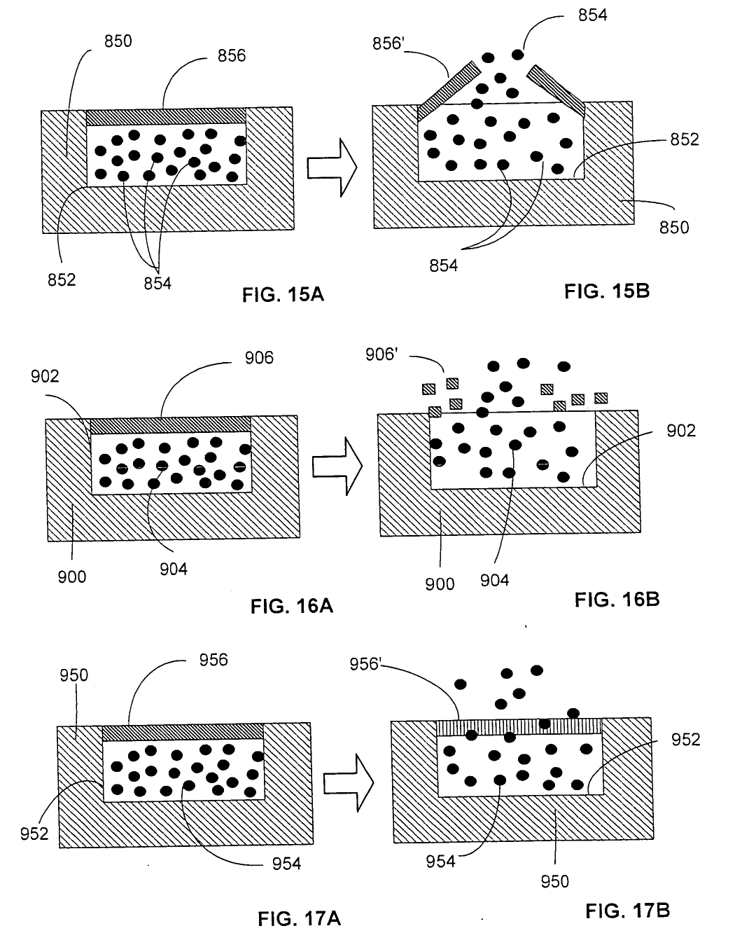

FIGS. 15A, 15B, 16A, 16B, 17A and 17B, illustrate several alternative embodiments of material release structures that include controllable barriers. In FIGS. 15A and 15 B, release structure 850 includes reservoir 852 containing stored deliverable material 854. As shown in FIG. 15A, while rupturable barrier 856 is intact, stored deliverable material 854 is contained within reservoir 852. As shown in FIG. 15B, when rupturable barrier 856 has been ruptured (as indicated by reference number 856′), deliverable material 854 may be released from reservoir 852. Rupturable barrier 856 may be ruptured by an increase of pressure in reservoir 852 caused by heating, for example, which may be controlled by response initiation circuitry. In another alternative shown in FIGS. 16A and 16B, release structure 900 includes reservoir 902 containing stored deliverable material 904. As shown in FIG. 16A, while degradable barrier 906 is intact, stored deliverable material 904 is contained within reservoir 902. As shown in FIG. 16B, degradation of degradable barrier 906 to degraded form 906′ causes stored deliverable material 904 to be released from reservoir 904. FIGS. 17A and 17B depict release structure 950 including reservoir 952 containing stored deliverable material 954. FIG. 17A, shows barrier 956, which has a controllable permeability, in a first, impermeable state, while FIG. 17B shows barrier 956 in a second, permeable state (indicated by reference number 956′). Stored deliverable material 954 passes through barrier 956′, when it is in its permeable state, and is released. Rupturable barriers as described above may be formed from a variety of materials, including, but not limited to, metals, polymers, crystalline materials, glasses, ceramics, semiconductors, etc. Release of materials through rupture or degradation of a barrier is also described in U.S. Pat. No. 6,773,429, which is incorporated herein by reference. Semipermable barriers having variable permeability are described, for example, in U.S. Pat. No. 6,669,683, which is incorporated herein by reference. Those of skill in the art will appreciate that barriers can be formed and operated reversibly through multiple release cycles, in addition to the single-release functionality available from a rupturable barrier.

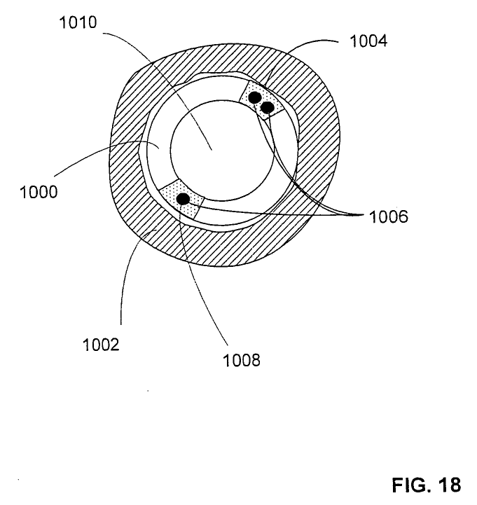

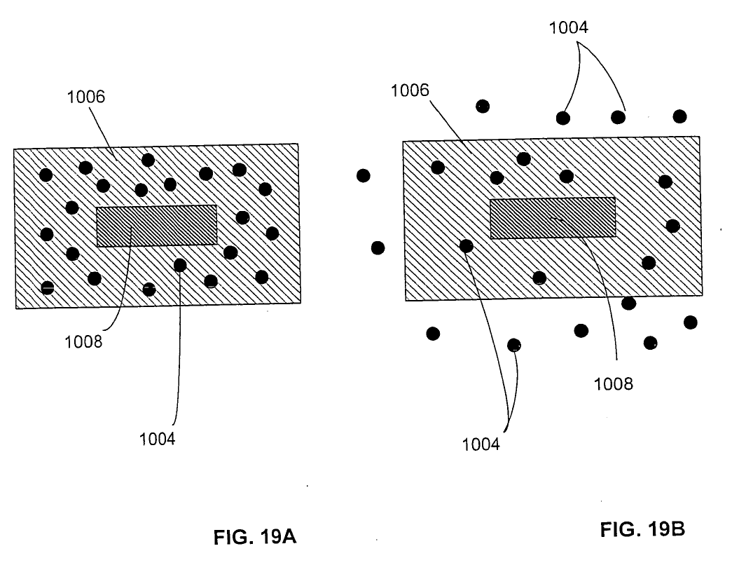

FIG. 18 depicts another embodiment of a lumenally-active device 1000 in a lumen containing structure 1002. Lumenally-active device 1000 includes stored deliverable material 1004 dispersed in a carrier material 1006. Stored deliverable material 1004 may be released from carrier material 1006 by release mechanism 1008 upon activation of release mechanism 1008. Released deliverable material 1004 may be released into central opening 1010 of lumenally-active device 1000, as well as into the volume defining the outermost portion of the lumen. FIGS. 19A and 19B depict in greater detail the release of stored deliverable material from the carrier material. In FIG. 19A, deliverable material 1004 is stored in carrier material 1006. Carrier material 1006 may be, for example, a polymeric material such as a hydrogel, and deliverable material is dispersed or dissolved within carrier material 1006. Release mechanism 1008 may be a heating element, for example a resistive element connected directly to response initiation circuitry, or an electrically or magnetically responsive material that may be caused to move, vibrate, heat, by an externally applied electromagnetic field, which in turn causes release of deliverable material 1004 from carrier material 1006, as shown in FIG. 19B. See, for example, U.S. Pat. Nos. 5,019,372 and 5,830,207, which are incorporated herein by reference. In some embodiments, an electrically or magnetically active component may be heatable by an electromagnetic control signal, and heating of the electrically or magnetically active component may cause the polymer to undergo a change in configuration. An example of a magnetically responsive polymer is described, for example, in Neto, et al, “Optical, Magnetic and Dielectric Properties of Non-Liquid Crystalline Elastomers Doped with Magnetic Colloids”; Brazilian Journal of Physics; bearing a date of March 2005; pp. 184-189; Volume 35, Number 1, which is incorporated herein by reference. Other exemplary materials and structures are described in Agarwal et al., “Magnetically-driven temperature-controlled microfluidic actuators”; pp. 1-5; located at: http://www.unl.im.dendai.ac.jp/INSS2004/INSS2004_papers/OralPresentations/C2.pdf or U.S. Pat. No. 6,607,553, both of which are incorporated herein by reference.

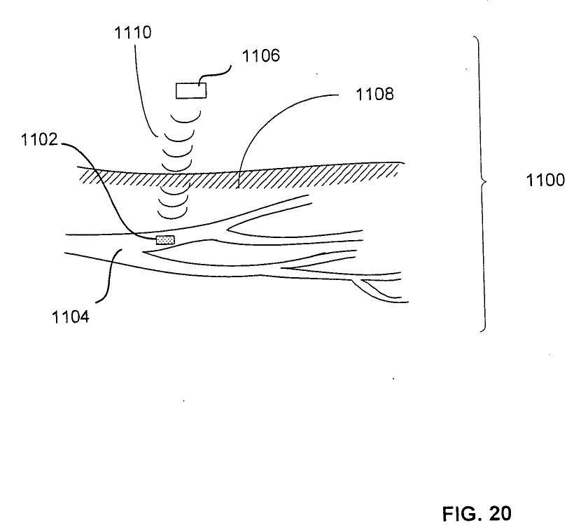

In some embodiments of lumenally-active devices or systems, a lumenally-active device may be a self-contained device that may be positioned in a body lumen and that includes all functionalities necessary for operation of the device. In other embodiments, as illustrated in FIG. 20, a lumenally-active system 1100 may include a lumenally-active device 1102 that may be placed in a body lumen 1104, and a remote portion 1106 that includes a portion of the functionalities of the lumenally-active system. In some embodiments, all functionalities essential for the operation of the lumenally-active device may be located on the lumenally-active device, but certain auxiliary functions may be located in remote portion 1106. For example, remote portion 1106 may provide for monitoring of the operation of the lumenally-active device or data collection or analysis. The remote portion may be located within the body of the subject at a distance from the lumenally-active device, or outside the body 1108 of the subject, as depicted in FIG. 20. Data and/or power signals may be transmitted between lumenally-active device 1102 and remote portion 1106 with the use of electromagnetic or acoustic signals 1110, or, in some embodiments, may be carried over electrical or optical links. In general, the remote portion may be placed in a location where there is more space available than within the body lumen, that is more readily accessible, and so forth. It is contemplated that a portion of the electrical circuitry portion of the lumenally-active system (which may include hardware, firmware, software, or any combination thereof) may be located in a remote portion. Methods of distributing functionalities of a system between hardware, firmware, and software at located at two or more sites are well known to those of skill in the art. The electrical circuitry portion of the lumenally-active system may include, but is not limited to, electrical circuitry associated with the sensor, response initiation circuitry, and electronics associated with the active portion. While the response initiation circuitry has been discussed within the context of electrical circuitry, it will be appreciated that in some embodiments other types of logic/circuitry may be used in place of or in addition to electrical circuitry, and the response initiation circuitry and other circuitry described herein is not limited to electrical circuitry. For example, fluid circuitry, chemo-mechanical circuitry, and other types of logic/circuitry may provide equivalent functionality and may be used in certain embodiments.

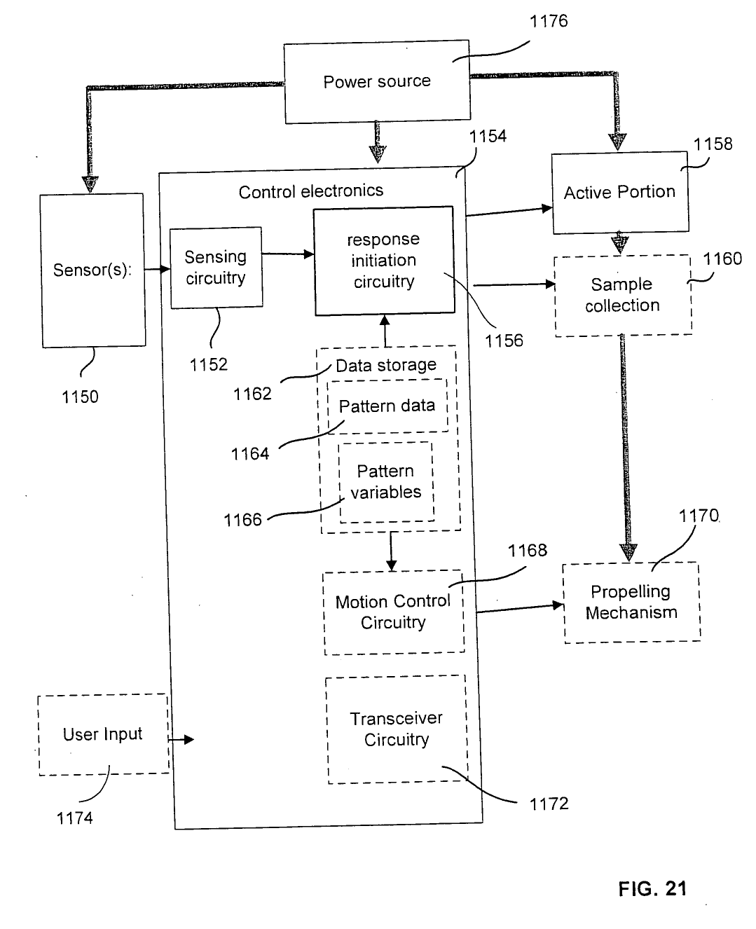

FIG. 21 is a block diagram illustrating in greater detail various electrical circuitry components of a lumenally-active system. As discussed herein, the electrical circuitry components may be located entirely on the structural element of a lumenally-active device, or may be distributed between the lumenally-active device and a remote portion as depicted in FIG. 19. The lumenally-active system may include one or more sensors 1150 for measuring or detecting a condition of interest. Sensing circuitry 1152 may be associated with sensors 1150. The lumenally-active system may include various control electronics 1154, including response initiation circuitry 1156. Response initiation circuitry 1156 provides response initiation signal to active portion 1158. In some embodiments, response initiation circuitry 1156 may also control sample collection portion 1160. Control electronics 1154 may also include data storage portion 1162, which may, for example, be used to store pattern data 1164 or pattern variables 1166. In some embodiments, control electronics 1154 may include motion control circuitry 1168 for controlling propelling mechanism 1170. Control electronics may include transceiver circuitry 1172, which provides for the transmission and reception of data and/or power signals between the lumenally-active device and a remote portion. A user input portion 1174 may provide for the input of user instruction, parameter, etc. to control electronics 1154. Finally, one or more power source 1176 may provide power to electrical components of the lumenally-active system.

Lumenally-active devices and systems according to various embodiments as described herein may include a power source, such as one or more batteries located on the lumenally-active device, possibly a microbattery like those available from Quallion LLC (http://www.quallion.com) or designed as a film (U.S. Pat. Nos. 5,338,625 and 5,705,293), which are incorporated herein by reference. Alternatively, the power source 1176 could be one or more fuel cell such as an enzymatic, microbial, or photosynthetic fuel cell or other biofuel cell (US20030152823A1; WO03106966A2 Miniature Biofuel cell; Chen T et al. J. Am. Chem. Soc. 2001, 123, 8630-8631, A Miniature Biofuel Cell, all of which are incorporated herein by reference), and could be of any size, including the micro- or nano-scale. In some embodiments, the power source may be a nuclear battery. The power source may be an energy-scavenging device such as a pressure-rectifying mechanism that utilizes pulsatile changes in blood pressure, for example, or an acceleration-rectifying mechanism as used in self-winding watches. In some embodiments, the power source may be an electrical power source located remote from the structural element and connected to the structural element by a wire, or an optical power source located remote from the structural element and connected to the structural element by a fiber-optic line or cable. In some embodiments, the power source may be a power receiver capable of receiving power from an external source, acoustic energy from an external source, a power receiver capable of receiving electromagnetic energy (e.g., infrared energy) from an external source.

The response initiation circuitry may include at least one of hardware, software, and firmware; in some embodiments the response initiation circuitry may include a microprocessor. The response initiation circuitry may be located in or on the structural element in some embodiments, while in other embodiments the response initiation circuitry may be at a location remote from the structural element.

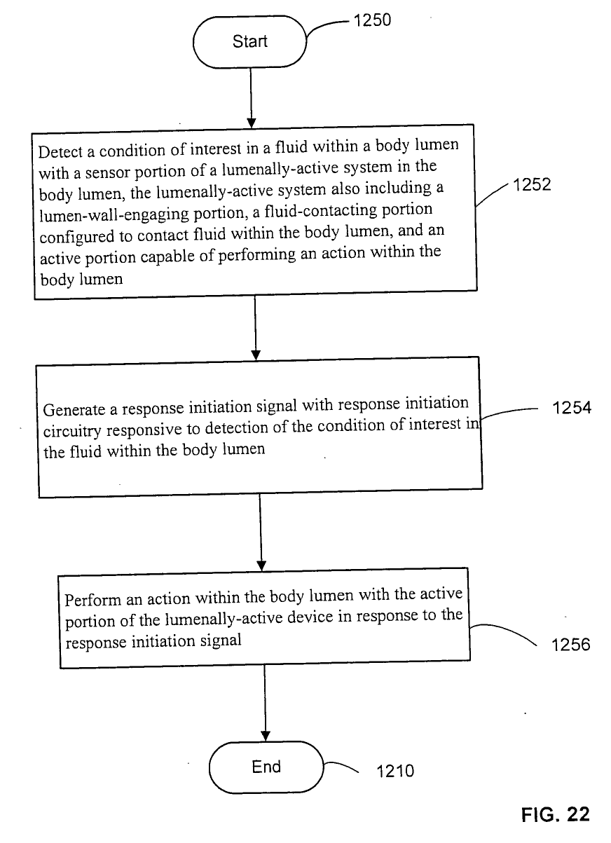

As shown in FIG. 22, a method of treating a body fluid using a lumenally-active device may include: detecting a condition of interest in a fluid within a body lumen with a sensor portion of a lumenally-active system in the body lumen, the lumenally-active system also including a lumen-wall-engaging portion, a fluid-contacting portion configured to contact fluid within the body lumen, and an active portion capable of performing an action within the body lumen, at step 1252; generating a response initiation signal with response initiation circuitry responsive to detection of the condition of interest in the fluid within the body lumen, at step 1254; and performing an action within the body lumen with the active portion of the lumenally-active device in response to the response initiation signal, at step 1256.

Detecting a condition of interest in a body fluid in the body lumen may include detecting a temperature, detecting a pressure, detecting a fluid flow, detecting an optical absorption, optical emission, fluorescence, or phosphorescence, detecting an index of refraction at at least one wavelength; detecting an acoustic signal, detecting an electrical resistivity, detecting a density or sound speed, detecting a pH, detecting an osmolality, detecting the presence of an embolism, detecting the presence (or absence) of an object (such as a blood clot, a thrombus, an embolus, a plaque, a lipid, a kidney stone, a dust particle, a pollen particle, a gas bubble, an aggregate, a cell, a specific type of cell, a cellular component or fragment, a collection of cell, a gamete, a pathogen, or a parasite), or detecting the presence (or absence) of a substance such as a biological marker, an antibody, an antigen, a peptide, a polypeptide, a protein, a complex, a nucleic acid, a cell (and, in some cases, a cell of a particular type, e.g. by methods used in flow cytometry), a cellular component, an organelle, a gamete, a pathogen, a lipid, a lipoprotein, an alcohol, an acid, an ion, an immunomodulator, a sterol, a carbohydrate, a polysaccharide, a glycoprotein, a metal, an electrolyte, a metabolite, an organic compound, an organophosphate, a drug, a therapeutic, a gas, a pollutant, or a tag, for example.

The step of performing an action within the body lumen with the active portion of the lumenally-active device in response to the response initiation signal may include activating a heating element, activating a cooling element, activating a material release portion, activating a material retrieval or sequestering portion, activating an analytic portion, activating an electromagnetic radiation source, activating an acoustic energy source, activating a pressure-generating element, activating a traction-generating element, activating a flow-modulating element capable of modulating the flow of fluid through at least a portion of the body lumen, activating a separator capable of at least partly removing specific components from at least a portion of the fluid, activating a catalytic portion to expose a catalytic surface to at least a portion of the fluid, activating an electric field source to apply an electric field to the fluid, activating a magnetic field source to apply a magnetic field to the fluid removing, modifying, or destroying at least a portion of the material of interest, or capturing at least a portion of the material of interest. In some embodiments, the presence of the material of interest may be desired, and if the absence (or a deficiency) of the material of interest is detected, performing an action within the body lumen with the active portion of the lumenally-active device may include adding the material of interest.

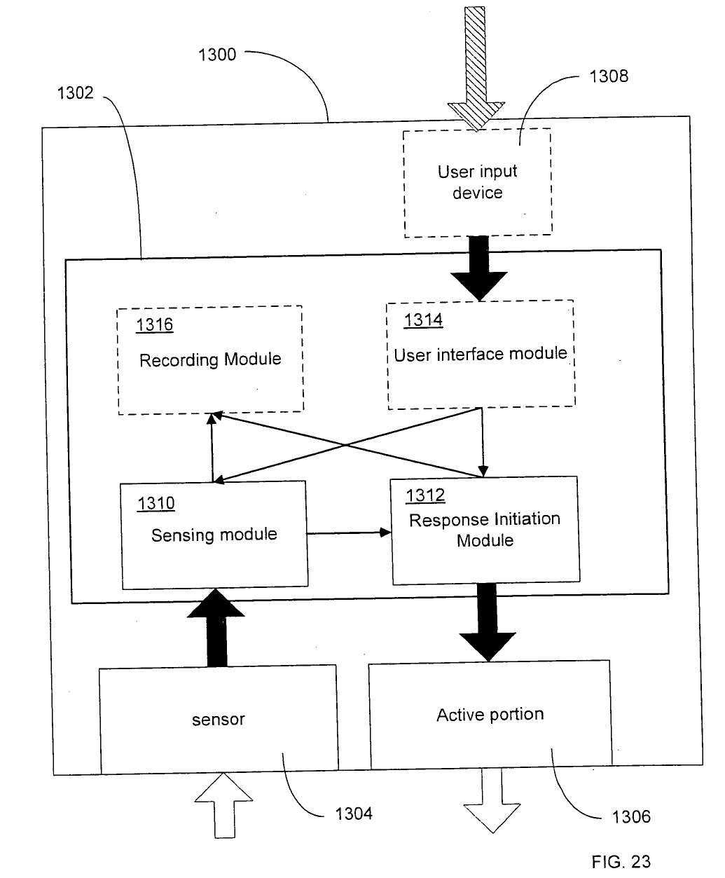

Lumenally-active devices and systems as described herein may be operated under the control of software. FIG. 23 illustrates in schematic form a lumenally-active device 1300. Components of lumenally-active device 1300 within box 1302 may be operated in whole or in part under software control. Lumenally-active device 1300 also includes components that may be primarily hardware-based, e.g., sensor 1304, active portion 1306, and, optionally, user input device 1308. Hardware-based devices may include components that are electrical, mechanical, chemical, optical, electromechanical, electrochemical, electro-optical, and are not limited to the specific examples presented herein. For example, software for operating a lumenally-active device may include a sensing module 1310 capable of receiving and processing a sense signal from a sensor portion 1304 of a lumenally-active system in a body lumen, the lumenally-active system also including a lumen-wall-engaging portion, a fluid-contacting portion configured to contact fluid within the body, lumen, and an active portion 1306 capable of performing an action within the body lumen, and producing as output one or more sense parameters; and a response initiation module 1312 capable of receiving as input the one or more sense parameters and generating as output a response initiation signal to cause the performance of an action within the body lumen with the active portion 1306 of the lumenally-active system.

The sensing module 1310 may be configured to receive the sense signal from the sensor 1304 substantially continuously, or the sensing module 1310 may include software code for controlling polling of the sense signal from the sensor portion to detect the presence of a condition of interest in the body lumen. In another alternative, the sensing module 1310 includes interrupt-driven software code responsive to an interrupt signal from the sensor portion to begin receipt and processing of the sense signal.

The sensing module 1310 may be configured to process the sense signal to determine the presence of the condition of interest. For example, the sensing module may be configured to process the sense signal by any of various signal processing methods, including, for example, filtering, signal amplification, windowing, noise reduction, clutter reduction, signal averaging, feature detection, time-domain analysis, frequency-domain analysis, feature extraction, comparison, sorting, reduction, and endpoint determination to determine the presence of the condition of interest. The sensing module 1310 may be configured to detect a critical value in the sense signal, the critical value indicative of the presence of a condition of interest in the body lumen.

The response initiation module 1312 may be configured to calculate the response initiation signal based at least in part upon at least one of the one or more sense parameters, or the response initiation module 1312 may be configured to generate the response initiation signal from a stored function. In some embodiments, the response initiation module may be configured to calculate the response initiation signal based at least in part upon at least one or more stored constants.

In some embodiments, the software may include a user interface module 1314 configured to receive user input of one or more user-enterable parameters from a user interface device 1308. In some embodiments, the software may include a recording module 1316 configured to record one or more values from the lumenally-active device over a recording interval. Recording module 1316 may also perform some processing of the information. In some embodiments, at least a portion of the one or more values may be sense signal values from sensor 1304. At least a portion of the one or more values may be sense parameter values. In some embodiments, at least a portion of the one or more values may be values corresponding to the action performed by the active portion of the lumenally-active system. In some embodiments, at least a portion of the one or more values may be response initiation signal values, corresponding to the response or action that is to be produced by active portion 1306. In some embodiments, a signal from active portion 1306 corresponding to the action produced by active portion 1306 may be recorded by recording module 1316. At least a portion of the one or more values may be response initiation signal values.

If the software includes a recording module, the response initiation module may be configured to generate the response initiation signal based at least in part upon one or more values received from the recording module. If the software includes a user-interface module, the response initiation may be is configured to generate the response initiation signal based at least in part upon one or more user-enterable parameters received from the user interface module.

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

In a general sense, those skilled in the art will recognize that the various embodiments described herein can be implemented, individually and/or collectively, by various types of electromechanical systems having a wide range of electrical components such as hardware, software, firmware, or virtually any combination thereof; and a wide range of components that may impart mechanical force or motion such as rigid bodies, spring or torsional bodies, hydraulics, and electro-magnetically actuated devices, or virtually any combination thereof. Consequently, as used herein “electromechanical system” includes, but is not limited to, electrical circuitry operably coupled with a transducer (e.g., an actuator, a motor, a piezoelectric crystal, etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), electrical circuitry forming a communications device (e.g., a modem, communications switch, or optical-electrical equipment), and any non-electrical analog thereto, such as optical or other analogs. Those skilled in the art will recognize that electromechanical as used herein is not necessarily limited to a system that has both electrical and mechanical actuation except as context may dictate otherwise. Non-electrical analogs of electrical circuitry may include fluid circuitry, electromechanical circuitry, mechanical circuitry, and various combinations thereof.

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

One skilled in the art will recognize that the herein described components (e.g., steps), devices, and objects and the discussion accompanying them are used as examples for the sake of conceptual clarity and that various configuration modifications are within the skill of those in the art. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1.-47. (canceled)

48. The system of claim 139, wherein the power source is a battery or microbattery.

49. The system of claim 139, wherein the power source is a fuel cell or biofuel cell.

50. The system of claim 139, wherein the power source is a nuclear battery.

51. The system of claim 139, wherein the power source is an energy-scavenging device.

52. The system of claim 51, wherein the energy-scavenging device includes a pressure-rectifying mechanism.

53. The system of claim 51, wherein the energy-scavenging device includes an acceleration-rectifying mechanism.

54. The system of claim 139, wherein the power source is an electrical power source located remote from the structural element and connected to the structural element by a wire

55. The system of claim 139, wherein the power source is an optical power source located remote from the structural element and connected to the structural element by a fiber-optic line or cable.

56. The system of claim 139, wherein the power source is a power receiver capable of receiving power from an external source.

57. The system of claim 139, wherein the power source is a power receiver capable of receiving acoustic energy from an external source.

58. The system of claim 139, wherein the power source is a power receiver capable of receiving electromagnetic energy from an external source.

59. The system of claim 139, wherein the response initiation circuitry is located in or on the structural element.

60. The system of claim 139, wherein the response initiation circuitry includes a microprocessor.

61. The system of claim 139, wherein the response initiation circuitry includes at least one of hardware, software, and firmware.

62. The system of claim 139, wherein the response initiation circuitry is at a location remote from the structural element.

63.-132. (canceled)

133. The software of claim 140, wherein at least a portion of the one or more values are sense signal values.

134. The software of claim 140, wherein at least a portion of the one or more values are sense parameter values.

135. The software of claim 140, wherein at least a portion of the one or more values are values corresponding to at least one action performed by the active portion of the lumenally-active system.

136. The software of claim 140, wherein at least a portion of the one or more values are response initiation signal values.

137. The software of claim 140, wherein the response initiation module is configured to generate the response initiation signal based at least in part upon one or more values received from the recording module.

138. (canceled)

139. A lumenally-active system, comprising:
a structural element configured to fit within at least a portion of a body lumen, the structural element including:
a lumen-wall-engaging portion; and
a fluid-contacting portion configured to contact fluid within the body lumen;
a sensor capable of detecting a condition of interest in the fluid;
response initiation circuitry operatively connected to the sensor and configured to generate a response initiation signal upon detection of the condition of interest in the fluid by the sensor;
an active portion operatively connected to the response initiation circuitry and capable of producing a response upon receipt of the response initiation signal; and
a power source configured to supply power to one or more electrical components of the lumenally active system.

140. Software for operating a lumenally-active device, comprising:
a sensing module capable of receiving and processing a sense signal from a sensor portion of a lumenally-active system in a body lumen, the lumenally-active system also including a lumen-wall-engaging portion, a fluid-contacting portion configured to contact fluid within the body lumen, and an active portion capable of performing an action within the body lumen, and producing as output one or more sense parameters;
a recording module configured to record one or more values from the lumenally-active device over a recording period; and
a response initiation module capable of receiving as input the one or more sense parameters and generating as output a response initiation signal to cause the performance of an action within the body lumen with the active portion of the lumenally-active system.
